(19) United States
(12) Patent Application Publication
(10) Pub. No.: US 2023/0187080 A1
(43) Pub. Date: Jun. 15, 2023

(54) Automation of Data Categorization for People with Autism

(71) Applicant: Alexander Santos Duvall, Magnolia, TX (US)

(72) Inventor: Alexander Santos Duvall, Magnolia, TX (US)

(73) Assignee: Alexander Santos Duvall, Magnolia, TX (US)

(21) Appl. No.: 18/047,649

(22) Filed: Oct. 19, 2022

(51) Int. Cl. G16H 50/70; G06F 16/65

(52) U.S. Cl. CPC G16H 50/70; G06F 16/65

(57) ABSTRACT

A custom artificial intelligence (AI) data categorization system and method is described for gathering and categorizing data that would overstimulate people with autism. Overstimulation is to be determined by our end-users' preferences. Our end-users will listen to a set of audio files and categorize them with: “Calm”, “Anxious”, or “Overstimulated”. The datasets presented to the end-user are randomly selected from data clusters that represent audio files with similar sounds based off a select set of attributes. Upon categorization, the selected set of attributes will be saved in a directory with its categorization saved in a database.

### FIELD

The present disclosure generally relates to a process for individuals with autism to classify auditory data.

### BACKGROUND OF THE INVENTION

Autism Spectrum Disorder (ASD) affects about 54 million people in the US, and it is set to grow upwards by about 64,000 a year. Though it is unclear what causes ASD, it is apparent that the presence of ASD has tremendously increased in the United States, and it is partly driven by an increased notice in childhood developmental delay which may include social or language deficits. As ASD has been researched over the last few decades more symptoms have been associated with it, and of the most common are meltdowns and overstimulation.

A meltdown is an extreme response to something that is upsetting, a stressor. These stressors may be, but not limited to, a sensory, emotional, or informational overload, overly difficult tasks or performance demands, unexpected life or environmental changes, or typical adult stressors like work demands, family or money. All of these stressors may be contributing factors or causes to a meltdown in some with ASD.

It should be known that a meltdown is not a tantrum, and they are not limited to just those with ASD. Each individual with ASD will exhibit different signs for a meltdown, and once a meltdown happens it cannot be stopped while it is ongoing. During a meltdown, an individual with Autism may become easily angered or violent, cover their ears to prevent further overstimulation, revert to self-harm, or scream.

Each person with autism may have a different stressor that leads to a meltdown, but a common one is overstimulation due to sound. These overstimulating sounds may come from vehicles, large crowds, loud noises, or anything a person with ASD finds uncomforting. These range of overstimulating sounds can be unique to each user, and there is no process that attempts to document what sounds may overstimulate a person with ASD. This present invention aims to use a process to gather information on what each person finds overstimulating.

### BRIEF SUMMARY OF THE INVENTION

A special purpose data categorization process of audio files for determining what sounds people with ASD find overstimulating is described herein. The process is made up of at least one audio file that will represent a cluster of other audio files. These audio files are clustered based off similarity regarding their Mel Spectrogram and Fourier signal attributes, or they are clustered based off of a set category determined prior to their use.

A user categorization tool is also described. To properly train our deep learning model to directly fit our user's needs, our users will be asked to classify sets of data clusters based on if they feel calm, anxious, or overstimulated after listening to it. Throughout the process, the user will be given intermittent breaks based on their input or manual intervention by the user. The user may stop the process and come back at a better time; the process will start where it stopped from the last time the user was on. Upon gaining the users categorization, results will be averaged if more than one audio file was listened to for one audio cluster.

A data storing methodology is also described. Upon each iteration the user classifies a sound for categorization, data is stored and encrypted in a database for record keeping. Initially, this data is sent to our API server on the cloud and then the data is properly rerouted to a database. The incoming data to be stored is encrypted prior to being stored in the database. This data may also be retrieved through a request from the API server. Upon retrieval, this data is decrypted and sent to the source destination IP address from the API server for use.

### DETAILED DESCRIPTION

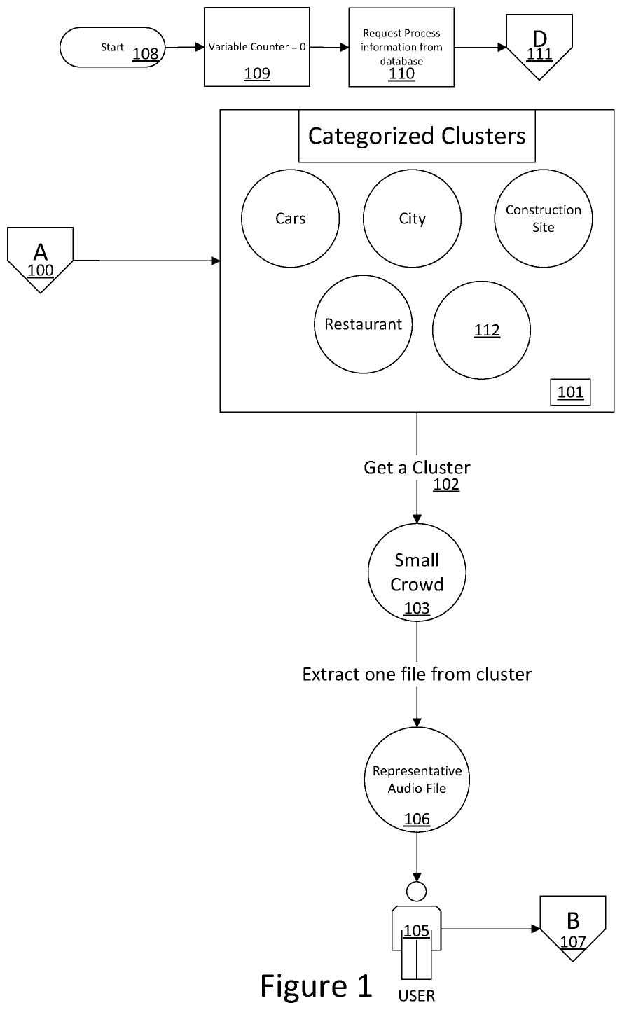

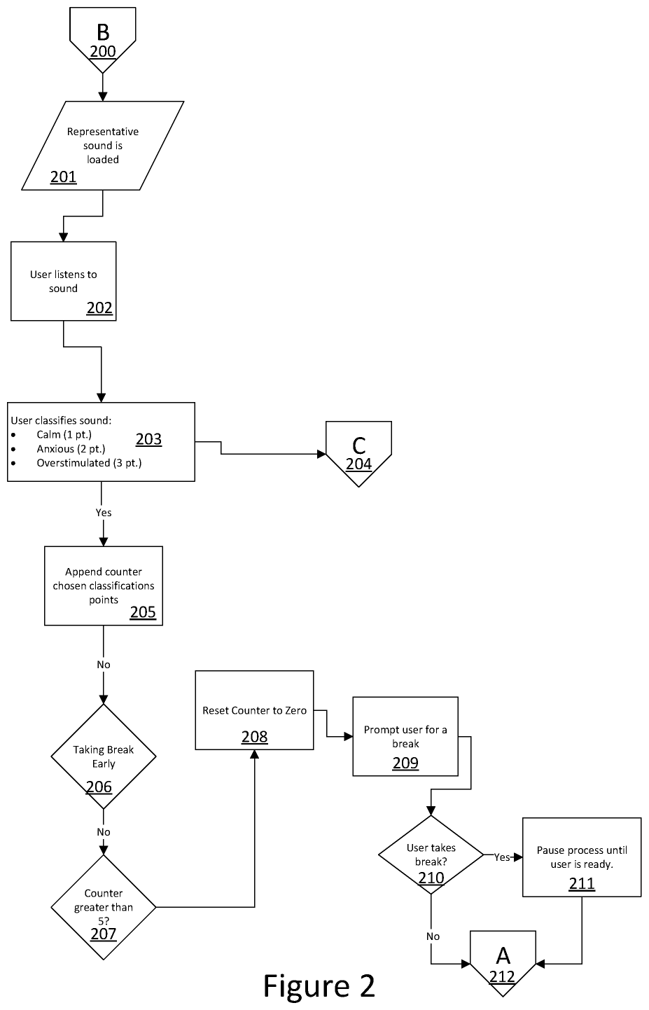

FIG. 1 shows a block diagram of the retrieval of cluster data for the categorization process, with the start of the process at FIG. 1 108. Upon starting the process, a counter variable is declared and initialized 109. Upon initialization, data is requested from the server 110 to retrieve information about the process if it was started prior. This request goes to reference D 111, where upon completion it will loop back to reference A 100. In the process, audio files are already clustered based off audio similarities, but they may not be categorized. For instance, some clusters may have already been labeled but others will just be grouped together without a label. This can be seen in FIG. 1 101 showing 5 clustered audio groups including an uncategorized cluster 112. This uncategorized cluster will hold no categorization until it is given one, but it will hold audio file associations from the database. Of these groups, one of them will be chosen 102 from the database. From the chosen cluster 103, a random audio file is pulled and downloaded, but not saved, 106 onto the user's device 105. Reference B 107 represents the move into FIG. 2 showing the categorization process.

Reference B in FIG. 2200 is the continuation of reference B in FIG. 1 107. Upon this continuation, the downloaded sound is loaded into the code 201, and the user listens to the loaded sound 202. Once the user listens to the sound, they have the choice of categorizing it as “Calm”, “Anxious”, or “Overstimulated” 203. Each of these categorizations respectively carries a weight of one, two, and three. After categorization, the variable “counter” 109, initialized in FIG. 1 with a value of 0 as an 8-bit integer, is appended by the number of points the user's categorization carried 205. For instance, if the user classified the audio sound as “Overstimulated”, then the variable “counter” would append by 3 points. This counter is used to determine if, and when, the user should be prompted to take a break. After the counter is appended 205 by the classification's corresponding value, the user's status is checked to see if they are taking a break from the process 206. If the user is not taking a break, then the user's counter value is checked 207. If the user's counter value is greater than 5, then the counter will be reset to zero 208, and the user will be prompted for a break 209. The user's decision is checked in the decision block 210. If the user decides to take a break, then the process will pause until the user is ready to resume 211. Once the user decides to go forward, they will return to the beginning of the process defined by reference A in FIG. 1 212 which is linked to reference A in FIG. 1 100. However, if the user decides not to take a break, then the process will restart from reference A in FIG. 2 212 to reference A in FIG. 1 100.

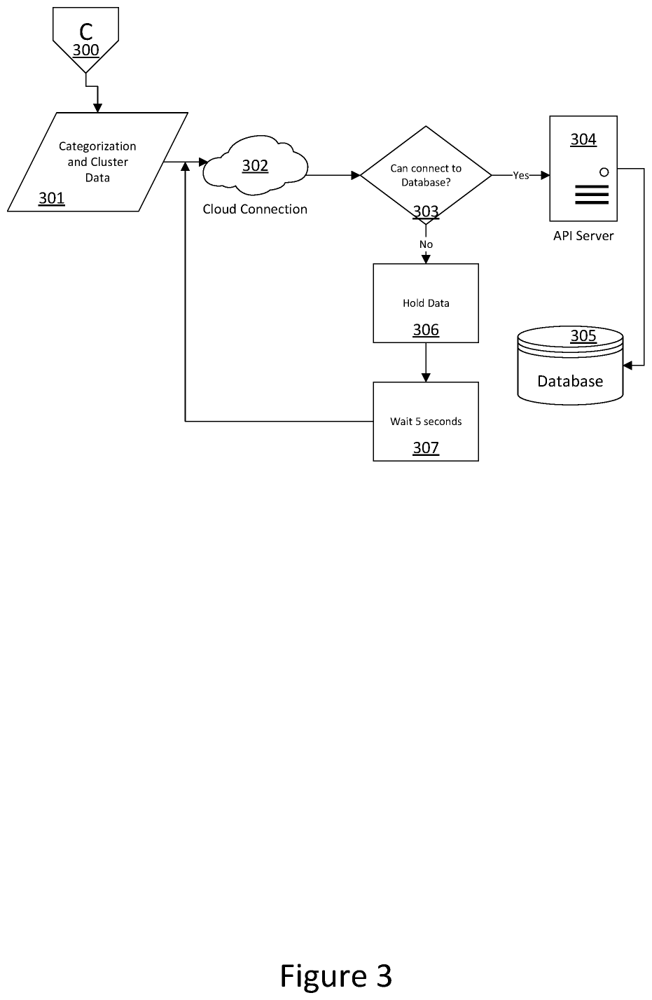

Upon classification of the audio sound FIG. 2 203 a background process for saving data is started through reference C in FIG. 2 204 which connects to reference C in FIG. 3 300. Starting from FIG. 3 300, FIG. 3 represents the data saving process. Block 301 represents the user input from FIG. 2 203 which includes the audio file's primary key as given by the database, the classification given from the user for the audio fie, and the user's primary key as given by the database. After the retrieval of data, a connection from the user's device to the selected cloud service 302 is attempted, and if the user is able to communicate with the database 303, then the data is sent to the API server 304 which is then rerouted to the database 305. However, if a connection cannot be forged with the cloud from the user's device, then the data is appended to a list where other unsent data are stored 306. Once the data is stored, the background thread will wait for 5 seconds 307 before attempting another connection to the cloud 302. Upon a successful cloud connection, the data contained inside the list holding unsent data will be sent to the API server where it will be rerouted to the database and stored. Anytime while the background process is running, the list holding the data can be modified,

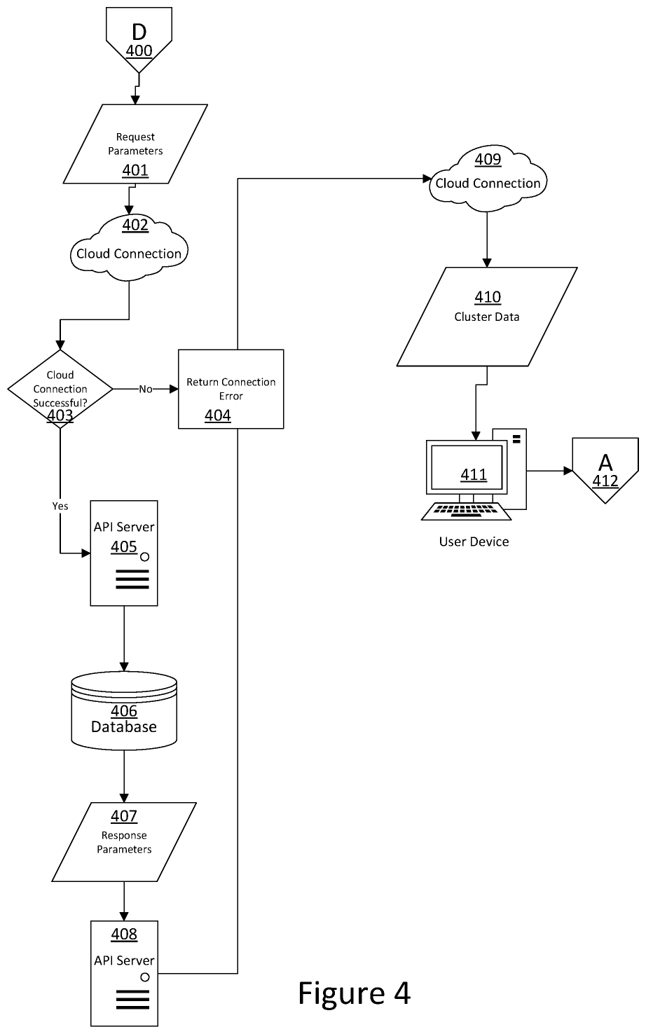

FIG. 4 represents the retrieval process for the database. If applicable, the user may choose to continue where they left off from the process given that they stopped before they finished the process. At the start of the process in FIG. 1 108 a request is made to the database from a chosen cloud service 402; FIG. 1 111 connects to diagram FIG. 4 400 where the request parameters 401 are sent. Given that a connection to the cloud 402 is not successful, as determined by block 403 the process returns a connection error, and the process is stopped 404. In the event of a successful connection, the parameters are sent to the API server 405 where a command is sent to the database. The database 406 receives this command and returns the necessary information 407, if any, to the API Server 408. The API Server will then respond to the initial source, through the chosen cloud service 409, that made the request 411 with the data 410 needed. The data sent back to the user's device 411 includes the returned primary keys of the Clusters that have yet to be completely categorized by the user for overstimulation. Once the user has retrieved the information it goes back to reference A FIG. 1 100 from FIG. 4 412.

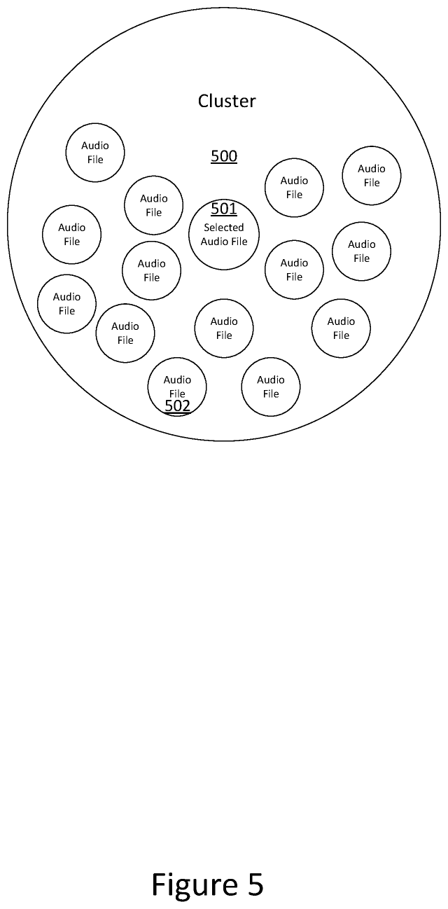

FIG. 5 is meant to visually represent the clustered audio files. The cluster 500 shown encompasses all audio files associated with it. Of all the audio files, one or more may be randomly selected to represent the cluster. In FIG. 5 the green reference 501 is the randomly selected audio file for the cluster 500. All other elements inside the cluster 500 labeled “Audio File”, like 502, are unselected audio files, but they are still associated to the cluster 500.

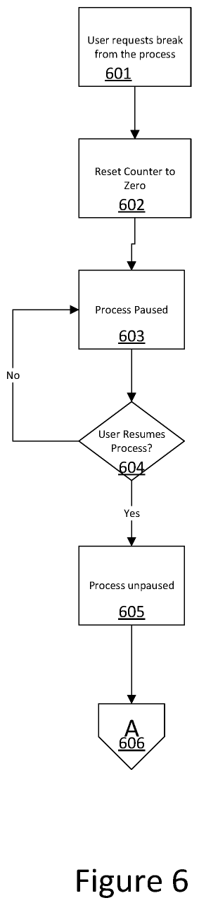

FIG. 6 represents the manual break process for initiating a manual break from the user. The user may start the break at any time during the data categorization process 601, and, except for the cloud processes outlined in FIG. 3 and FIG. 4, it will pause the categorization process at whatever stage it may be on the user's device 603. Prior to pausing the process, the counter variable is set to the value zero 602. While the categorization process is paused, it is checked if the user has ended the break 604. If they have chosen to resume, the categorization process will be unpaused 605, and it will begin again through reference A 606 connected to reference A in FIG. 1 100.

What is claimed is:

1. A data categorization process of audio files for determining what sounds people with autism spectrum disorder find overstimulating, the process comprising:
A break based on the user's classification of an audio file;
An extraction of unique cluster information that pulls data from the database;
An extraction of data from a data cluster to act as the cluster's representative audio file;
A saving algorithm that is run on a separate background thread in parallel with the categorization process;
An extraction of cluster data from the database.

2. The process of claim 1 wherein user data of audio sound classification is saved after classification in a database.

3. The process of claim 1 further comprising the user data is kept in a list until a cloud connection can be made.

4. The process of claim 1 wherein the unique cluster data consists of uncategorized data clusters the user has yet to classify.

5. The process of claim 4 wherein the unique cluster data is returned as a list of primary keys from the database back to the user's device.

6. The process of claim 1 further comprising a break based on the user's classification is given when the counter in the code exceeds or is equal to 5.

7. The process of claim 6 wherein a given break resets the counter to zero.

8. The process of claim 1 wherein a break may be called by the user at any time during the process and the counter will be reset to zero.

9. The process of claim 1 wherein the extracted audio file from the cluster is randomly chosen from the entire batch.

10. The process of claim 9 wherein the selected audio file is returned to the device that made the request.

11. The process of claim 2 wherein the user data to be stored in the database includes the categorization of a representative audio file.

12. The process of claim 3 wherein the user data stored in the list includes the categorization of a representative audio file.

13. The process of claim 1 wherein the categorization of representative audio files will categorize the entire cluster.

14. The process of claim 13 wherein the categorization of an entire cluster is specific to the user only and not a permanent classification of an entire cluster for all users.
